(19) United States
(12) Patent Application Publication
(10) Pub. No.: US 2009/0297587 A1
(43) Pub. Date: Dec. 3, 2009

(54) HYDROGEL WOUND DRESSING AND ITS METHOD OF PREPARATION

(75) Inventors: Zhanshan Yang, Suzhou (CN); Zhigao Rao, Suzhou (CN); Ling Yue, Suzhou (CN); Shuqin Yang, Suzhou (CN); Nankang Zhu, Suzhou (CN)

Correspondence Address: FULWIDER PATTON LLP, HOWARD HUGHES CENTER, 6060 CENTER DRIVE, TENTH FLOOR, LOS ANGELES, CA, 90045, US

(73) Assignee: SUZHOU UNIVERSITY, Suzhou (CN)

(21) Appl. No.: 12/470,810

(22) Filed: May 22, 2009

(30) Foreign Application Priority Data
May 28, 2008 (CN) 200810122438.1

(51) Int. Cl. A61K 9/70

(52) U.S. Cl. 424/445

(57) ABSTRACT

An improved hydrogel wound dressing and its preparation is disclosed. The ingredients used in the dressing are PAAS of 20˜30 wt %, PVA of 2˜6 wt % and water of 64˜78 wt %. The hydrogel wound dressings were formed by dissolution, casting films, radiation synthesis and sterilization. Compared with conventional hydrogel dressings the improved dressings displayed an enhanced degree of swelling. Cell growth factors may be incorporated into the dressings to be released slowly in the wound.

### CROSS-REFERENCES TO RELATED APPLICATIONS

This application claims priority from Chinese Application No. 200810122438.1, filed May 28, 2008, the contents of which are fully incorporated by reference herein.

The present invention relates to a method for synthesizing a hydrogel wound dressing with an optimal swelling capacity.

### BACKGROUND

At present, medical treatment of wounds such as burns, scalds and trauma commonly uses a wet dressing in order to maintain a moist environment in the area of the wound for a long time. The wet dressing can alleviate pain and facilitate the healing of wounds. At present, the wound dressings mainly comprise three types, i.e. a general type, a biotype and a compound type. The compound types are mostly a hydrogel wound dressing, that is formed by crosslinking of polymer solution such as polyvinyl alcohol (PVA), Sodium Polyacrylate (PAAS), polyethylene oxide (PEO), poly(vinyl pyrrolidone) (PVP), polyacrylic acid, polyoxyethylene and polyacrylamide. Compared with the other two types of dressings, the hydrogel wound dressings show more satisfactory properties of hygroscopicity, gas and vapor permeability and biocompatibility. The polymers are readily available and inexpensive. Therefore hydrogel wound dressings and methods for their preparation have been widely studied and applied.

For example, Chinese Patent No. CN1065771C, the contents of which are incorporated herein by reference, discloses a preparation method of a medical hydrogel film, in which PEO and PVA were prepared by dissolving, casting films, cold and heat alternating process, and radiation synthesis. This hydrogel film has two deficiencies as a wound dressing. At first, the swelling ratio of the PEO/PVA wound dressing is only 1300%, so it does not fully absorb all of the wound effusion. Therefore dressings need to be changed every 2˜3 days, which not only increases the staff's work load but also uses more hydrogel films. It is possible that frequent changing of a wound dressing will increase the opportunity for wound infection, in particular larger skin wounds with a mass of effusion. Secondly, the cold and heat alternating process can consume a great deal of energy and make the preparation process quite complex.

### SUMMARY OF THE INVENTION

The present invention provides a medical hydrogel wound dressing of high hydroscopic property. The dressing also exhibits a high degree of swelling, which decreases the frequency for dressing changes.

The present invention also provides a simple preparation method of the hydrogel wound dressing.

The technical outline of the present invention is as follows:

(1) proportioning raw materials of the hydrogel wound dressing, which are

| | |
|---|---|
| Sodium Polyacrylate | 20~30 wt % |
| Polyvinyl Alcohol | 2~6 wt % |
| Distilled Water | 64~78 wt % |

(2) Dissolution and casting films

Mixing the above three components, heating the mixture to dissolve them to form a completely homogeneous solution, then pouring the solution into Petri dishes and allowing them to cool down to form casting films.

(3) Radiation synthesis

To synthesize the hydrogel wound dressing, a radiation crosslinking process is used. The casting films in the dishes are irradiated at a dose rate of 15 kGy/pass and the total dose of 30˜90 kGy using an electron accelerator.

(4) Preserving the hydrogel wound dressing at 0˜5° C. after encapsulation and sterilization.

The radiation crosslinking process in step (3) is an existing technology. 60Co γ-ray radiation also can be applied in the present invention.

In this procedure, the thickness of the casting films in step (2) ranges from 0.5 mm to 1.5 mm.

During this preparation, some drugs such as cell growth factors can be incorporated into the hydrogel dressings. These drugs will be released slowly into the lesions and accelerate the wound healing.

### THE ADVANTAGES OF THE INVENTION

Compared with existing hydrogel wound dressings, the advantages of the invention are as follows:

1. The medical hydrogel wound dressings, made by the preparation method of the present invention, have an excellent degree of swelling i.e. hydroscopic property. The hydrogel wound dressings can absorb a large wound effusion and retaining the wound moisture well, so that the frequency of dressing change is decreased, which not only reduces the staff's work load but also the number of dressings required. The hydrogel wound dressings can keep the area of wounds moist for a long time and accelerate the wound healing.

2. The medical hydrogel wound dressing formed by the preparation method of the present invention has an excellent tensile strength, so it is not easily torn when it is applied to a wound. It will not bind to a wound and cause secondary wound damage when it is changed. It can protect the wound from infection by external microbial pathogens. It has no cytotoxicity, acute toxicity, irritability, and it will not irritate the skin, so the medical hydrogel wound dressings show perfect histocompatibility. Therefore, the medical hydrogel dressing is an attractive and marketable product.

3. The swelling ratio of existing PEO/PVA wound dressings amounts to 1300%. The swelling ratio of the hydrogel film made of Sodium Polyacrylate alone is from 1911% to 2339%. However the swelling ratio of the medical hydrogel dressing of the present invention can reach 57210%. Hence the hydroscopic property of the medical hydrogel dressings of the present invention is a marked improvement in the international field of the hydrogel wound dressing.

### DETAILED DESCRIPTION OF THE PREFERRED EMBODIMENTS

The present invention will now be illustrated by the following examples, which are illustrative of, but not restrictive of, the present invention.

### EXAMPLE 1

### Preparation of a Medical Hydrogel Wound Dressing Comprising of Four Successive Procedures

1) According to the weight rate of PAAS: PVA=20˜30:2˜6, mix them with double distilled water to form a polymer suspension of 27˜31 wt. %.

2) Dissolution and casting films

The suspension is heated at 120° C. for 2 hours in an autoclave to dissolve the polymers. Then the completely homogeneous solution is poured into Petri dishes and allowed to cool down to form casting films. The thickness of the casting films is 1 mm.

3) Radiation synthesis

To form the hydrogel wound dressing, the casting films are irradiated at an energy of 2.45 MeV, and beam current of 14 mA at a dose rate of 15 kGy/pass, generated from an electron accelerator. The total dose is 30˜90 kGy.

4) Preservation at 4° C. after sterilization.

The hydrogel wound dressing is sealed within an airproof silver paper or plastic bag, and sterilized by 60 Co y-ray radiation, and then stored at 4° C.

100 ng of vascular endothelial growth factor per unit area can be incorporated into the hydrogel dressings to be released slowly into the local wound. The healing of a radioactive burn induced in experimental animals is accelerated by 6˜8 days compared with a conventional petroleum based gauze dressing such as Vaseline®.

In the above method, the ratio of the materials and the radiation condition can be changed. Hence many kinds of hydrogel dressings can be prepared with these different properties.

1. Swelling ratio

The degree of swelling is defined as the ratio of the water mass of swollen gel to the dry gel's mass. The dry gel mass was determined by drying five gel samples to constant weight in a vacuum at 80° C. The swollen gel was measured by immersing the dry gels in double distilled water at room temperature (25° C.) for 24 hours. The results are shown in Table 1.

TABLE 1
The degree of swelling of the hydrogel dressing (×100%)

| Absorbed | Ratio of materials (PAAS:PVA) | | | |
|---|---|---|---|---|
| Dose (kGy) | 25:2 | 25:4 | 25:6 | 25:0 |
| 30 | 344.7 ± 25.4 | 239.1 ± 23.4 | 249.8 ± 18.8 | |
| 45 | 572.1 ± 44.9 | 367.8 ± 30.8 | 354.1 ± 25.8 | 19.11 ± 3.30 |
| 60 | 354.3 ± 4.8 | 292.8 ± 10.3 | 283.0 ± 7.5 | 22.97 ± 4.97 |
| 75 | 306.7 ± 24.5 | 246.4 ± 12.9 | 239.8 ± 4.0 | 24.29 ± 3.70 |
| 90 | 251.8 ± 21.8 | 198.0 ± 5.3 | 193.1 ± 10.5 | 23.39 ± 7.20 |

As shown in Table 1, the preparation of the invention hydrogel wound dressing was the same as that of the PAAS hydrogel film prepared from only Sodium Polyacrylate. However the degree of swelling of the former is 20˜30 times as much as the latter. The aforesaid hydrogel dressings are especially suitable to use on large wounds with high effusion such as burn, trauma, scald and so on.

2. Gel fraction

Five gel samples were dried until they reached constant weight. The dried samples were immersed in double distilled water at room temperature (25° C.) for 24 hours to extract the sol. The gels were dried. The gel fraction was defined as the ratio of the dried gel mass weight after extraction to the initial mass weight of the dried sample.

TABLE 2
Gel fraction of the hydrogel dressing (%)

| Absorbed | Ratio of materials (PAAS:PVA) | | |
|---|---|---|---|
| Dose (kGy) | 25:2 | 25:4 | 25:6 |
| 30 | 49.90 ± 6.38 | 44.74 ± 5.14 | 51.00 ± 2.94 |
| 45 | 60.01 ± 6.99 | 62.89 ± 4.82 | 65.15 ± 2.82 |
| 60 | 66.61 ± 8.75 | 69.74 ± 5.86 | 72.34 ± 5.14 |
| 75 | 69.60 ± 5.61 | 72.22 ± 1.65 | 74.94 ± 4.19 |
| 90 | 71.10 ± 6.70 | 74.68 ± 4.22 | 77.91 ± 4.98 |

3. Tensile strength

The hydrogel dressings were cut into a dumbbell shape. The total length of the dumbell was 59 mm and the width at each end was 10 mm. The narrower central region was 10 mm long and 7 mm wide. The tensile strength at breaking point was measured using a tension meter (SZL-200) with a crosshead speed of 100 mm/min at room temperature (25° C.) and air humidity of 60%.

TABLE 3
The tensile strength of the hydrogel dressing (MPa)

| Absorbed | Ratio of materials (PAAS:PVA) | | |
|---|---|---|---|
| Dose (kGy) | 25:2 | 25:4 | 25:6 |
| 30 | 0.337 ± 0.059 | 0.630 ± 0.045 | 0.956 ± 0.099 |
| 45 | 0.397 ± 0.026 | 0.711 ± 0.078 | 1.152 ± 0.111 |
| 60 | 0.418 ± 0.024 | 0.847 ± 0.061 | 1.041 ± 0.050 |
| 75 | 0.457 ± 0.034 | 0.952 ± 0.073 | 1.028 ± 0.067 |
| 90 | 0.476 ± 0.070 | 0.647 ± 0.104 | 0.889 ± 0.093 |

4. Tests of biocompatibility

The tests of biocompatibility were performed by the Center of Test and Assay of the Institute of Radiation Medicine of Soochow University.

The tests of biocompatibility comprised assays for intradermal stimulation, skin sensitization, acute toxicity and cytotoxicity. The results indicated that the hydrogel dressings have no skin stimulation and sensitization, no acute toxicity reactions and cytotoxicity.

The properties of the hydrogel wound dressings showed that they had the highest degree of swelling, an acceptable gel fraction, a very satisfactory mechanical strength and biocompatibility. Cell growth factors which are important to improve wound healing can be incorporated into it and released slowly. The hydrogel wound dressing is very effective for treating fire burns, scald and wounds, especially for large area skin wounds with a mass of exudate.

We claim:

1. A method for preparing a medical hydrogel wound dressing comprising the steps of:
(1) proportioning raw materials of the hydrogel wound dressing comprising:

| | |
|---|---|
| Sodium Polyacrylate | 20~30 wt % |
| Polyvinyl Alcohol | 2~6 wt % |
| Distilled Water | 64~78 wt % |

(2) mixing and heating the raw materials until dissolved to form a homogeneous solution, and then pouring the homogeneous solutions into dishes to cool down to form casting films;
(3) irradiating the casting films using an electron beam accelerator to a total dose of between 30-90 kGy at a dose rate of 15 kGy/pass to radiation synthesize the hydrogel wound dressing; and
(4) preserving the hydrogel wound dressing at 0˜5° C. after encapsulation and sterilization.

2. The method of preparing the medical hydrogel wound dressing according to claim 1, wherein the thickness of the casting films amounts to 0.5˜1.5 mm.

3. The method of preparing the medical hydrogel wound dressing according to claim 1, wherein the hydrogel wound dressings further contains cell growth factors.

4. A hydrogel wound dressings for clinical medicine obtained by the method of claim 1.
